(19) United States
(12) Patent Application Publication
(10) Pub. No.: US 2015/0226745 A1
(43) Pub. Date: Aug. 13, 2015

(54) PROSTATE CANCER MARKERS AND USES THEREOF

(71) Applicants: Tore SKOTLAND, (US); Alicia LLORENTE, (US); Kirsten SANDVIG, (US); OSLO UNIVERSITETSSYKEHUS HF, Nydalen (NO)

(72) Inventors: Tore Skotland, Nittedal (NO); Alicia Llorente, Oslo (NO); Kirsten Sandvig, Nittedal (NO)

(21) Appl. No.: 14/429,530

(22) PCT Filed: Feb. 28, 2013
(86) PCT No.: PCT/IB2013/000877

§ 371 Date: Mar. 19, 2015

Related U.S. Application Data
(60) Provisional application No. 61/703,496, filed on Sep. 20, 2012.

(51) Int. Cl. G01N 33/574; G01N 30/72; G01N 33/92

(52) U.S. Cl. CPC G01N 33/57434; G01N 33/92; G01N 30/72; G01N 2405/08; G01N 2405/10

(57) ABSTRACT

The present invention relates to compositions and methods for cancer diagnosis, research and therapy, including but not limited to, cancer markers. In particular, the present invention relates to lipids as diagnostic markers for prostate cancer.

### FIELD OF THE INVENTION

The present invention relates to compositions and methods for cancer diagnosis, research and therapy, including but not limited to, cancer markers. In particular, the present invention relates to lipids as diagnostic markers for prostate cancer.

### BACKGROUND OF THE INVENTION

Afflicting one out of nine men over age 65, prostate cancer (PCA) is a leading cause of male cancer-related death, second only to lung cancer (Abate-Shen and Shen, Genes Dev 14:2410 [2000]; Ruijter et al., Endocr Rev, 20:22 [1999]). The American Cancer Society estimates that about 241,740 American men will be diagnosed with prostate cancer and 28,170 will die in 2012.

Prostate cancer is typically diagnosed with a digital rectal exam and/or prostate specific antigen (PSA) screening. An elevated serum PSA level can indicate the presence of PCA. PSA is used as a marker for prostate cancer because it is secreted mainly by prostate cells. A healthy prostate will produce a stable amount—typically below 4 nanograms per milliliter of blood, or a PSA reading of “4” or less—whereas cancer cells produce escalating amounts that correspond with the severity of the cancer. A level between 4 and 10 nanograms of PSA per milliliter of blood may raise a doctor's suspicion that a patient has prostate cancer, while amounts above 50 may show that the tumor has spread elsewhere in the body.

When PSA or digital tests indicate a strong likelihood that cancer is present, a transrectal ultrasound is used to map the prostate and show any suspicious areas. Biopsies of various sectors of the prostate are used to determine if prostate cancer is present. Treatment options depend on the stage of the cancer. Men with a 10-year life expectancy or less who have a low Gleason number and whose tumor has not spread beyond the prostate are often treated with watchful waiting (no treatment). Treatment options for more aggressive cancers include surgical treatments such as radical prostatectomy, in which the prostate is completely removed (with or without nerve sparing techniques) and radiation, applied through an external beam that directs the dose to the prostate from outside the body or via low-dose radioactive seeds that are implanted within the prostate to kill cancer cells locally. Anti-androgen hormone therapy is also used, alone or in conjunction with surgery or radiation. Hormone therapy uses luteinizing hormone-releasing hormones (LH-RH) analogs, which block the pituitary from producing hormones that stimulate testosterone production. Patients must have injections of LH-RH analogs for the rest of their lives.

While surgical and hormonal treatments are often effective for localized PCA, advanced disease remains essentially incurable. Androgen ablation is the most common therapy for advanced PCA, leading to massive apoptosis of androgen-dependent malignant cells and temporary tumor regression. In most cases, however, the tumor reemerges with a vengeance and can proliferate independent of androgen signals.

The advent of prostate specific antigen screening has led to earlier detection of PCA and significantly reduced PCA-associated fatalities. However, the U.S. Preventive Services Task force recently decided to recommend against screening for PSA based on five recent trials all showing either a small or no benefit in prostate-cancer-specific mortality (Screening for prostate cancer: draft recommendation statement. Rockville, Md.: U.S. Preventive Services Task Force, Oct. 7, 2011). Furthermore, a major limitation of the serum PSA test is a lack of prostate cancer sensitivity and specificity especially in the intermediate range of PSA detection (4-10 ng/ml). Elevated serum PSA levels are often detected in patients with non-malignant conditions such as benign prostatic hyperplasia and prostatitis, and provide little information about the aggressiveness of the cancer detected. Coincident with increased serum PSA testing, there has been a dramatic increase in the number of prostate needle biopsies performed (Jacobsen et al., JAMA 274:1445 [1995]). This has resulted in a surge of equivocal prostate needle biopsies (Epstein and Potter J. Urol., 166:402 [2001]). Thus, development of additional biomarkers to supplement PSA screening is needed.

### SUMMARY OF THE INVENTION

The present invention relates to compositions and methods for cancer diagnosis, research and therapy, including but not limited to, cancer markers. In particular, the present invention relates to lipids as diagnostic markers for prostate cancer.

Embodiments of the present invention provide compositions, kits, and methods useful in the detection and screening of prostate cancer. For example, in some embodiments, the present invention provides methods of characterizing a prostate sample or diagnosing prostate cancer in a subject, comprising contacting a biological sample comprising an extracellular vesicle released from a prostate cell with a reagent for detecting the presence of one or more (e.g., two or more, three or more, four or more, five or more, or six or more lipids (e.g., the lipids described in Table 1, for example, a glycosphingolipid (e.g., hexosylceramide (HexCer), lactosylceramide (LacCer), Gb,3 and the gangliosides monosialotetrahexosylganglioside (GM1), Ganglioside GM2 (GM2), monosialodihexosylganglioside (GM3), and ganglioside GD1 (GD1)), sphingomyelin (SM), and cholesterol; and detecting the presence of the one or more lipids in said sample using an in vitro assay. In some embodiments, the in vitro assay comprises an immunoassay. In some embodiments, the assay is a mass spectrometry assay. In some embodiments, the assay is amplification (e.g., PCR) in combination with an immunoassay. In some embodiments, molecules (e.g., proteins that are not antibodies) bind to lipids and are then detected using a suitable analytical method (e.g., immunoassay or other analytical method). In some embodiments, the method further comprises the step of enriching the sample for the presence of extracellular vesicles. In some embodiments, the prostate cell is a prostate cancer cell. In some embodiments, the presence of the lipids in the sample is indicative of prostate cancer in the subject. In some embodiments, the sample is blood, serum, plasma, prostatic fluid, urine, or semen. In some embodiments, the reagent is an antibody that specifically binds to said one or more lipids.

Further embodiments provide the use of reagents for detecting the presence or amount of one or more (e.g., two or more) lipids in a biological sample for providing a diagnosis or prognosis related to prostate cancer. In some embodiments, the present invention provides methods for predicting a predisposition to prostate cancer in a subject, diagnosing a prostate cancer in a subject, predicting the likelihood of recurrence of prostate cancer in a subject, providing a prognosis for a subject with prostate cancer, or selecting a subject with cervical cancer for treatment with a particular therapy, comprising: (a) detecting the presence of one or more lipids in a sample from said subject using an in vitro assay and (b) comparing said one or more lipids in said sample with a reference, wherein an altered amount of said one or more lipids relative to said reference provides an indication selected from the group consisting of an indication of a predisposition of the subject to prostate cancer, an indication that the subject has prostate cancer, an indication of the likelihood of recurrence of the prostate cancer in the subject, an indication of survival of the subject, an indication of the likely outcome of treatment of the prostate cancer and an indication that the subject is a candidate for treatment with a particular therapy. In other embodiments, the present invention provides methods of screening for the presence of prostate cancer in a subject, comprising providing a biological sample from a subject and analyzing the sample for the presence or amount of one or more lipids in said sample, wherein the presence of the lipids in the sample is indicative of prostate cancer in said subject. In some embodiments, elevated amounts of one or more of glycosphingolipids, sphingomyelin, or cholesterol, in the biological sample as compared to a reference is indicative of prostate cancer. In some embodiments, the glycosphingolipids are selected from the group consisting of HexCer, LacCer, Gb3, GM1, GM2, GM3, and GD1. In some embodiments, lowered amounts of one or more of phosphatidylinositol, phosphatidylglycerol and phosphatidylcholine in the biological sample as compared to a reference is indicative of prostate cancer. In some embodiments, a lipid profile is determined for the patient sample and compared to a reference lipid profile. The lipid profile may preferably comprise the presence and/or relative amounts of one, two, three, four, five or more lipids in the sample, for example in a sample comprising or enriched for extracellular vesicles. For example, the lipid profile may comprise the presence and/or relative amounts of one, two, three, four, five or more of glycosphingolipids such as HexCer, LacCer, Gb3, GM1, GM2, GM3, GD1, sphingomyelin, cholesterol, phosphatidylinositol, phosphatidylglycerol and phosphatidylcholine. When compared to a reference profile, the increased amount of one or more of glycosphingolipids such as HexCer, LacCer, Gb3, GM1, GM2, GM3, and GD1, sphingomyelin, and cholesterol and/or the decreased amount of one or more of phosphatidylinositol, phosphatidylglycerol and phosphatidylcholine is considered to be an indication of a predisposition of the subject to prostate cancer, an indication that the subject has prostate cancer, an indication of the likelihood of recurrence of the prostate cancer in the subject, an indication of survival of the subject, an indication of the likely outcome of treatment of the prostate cancer and an indication that the subject is a candidate for treatment with a particular therapy.

In some embodiments, the in vitro assay is selected from the goup consisting of a mass spectrometry assay, a gas chromatography assay, a liquid chromatography assay, and immunoassay, and combinations thereof. In some embodiments, the methods further comprise the step of enriching said sample for the presence of extracellular vesicles. In some embodiments, the sample comprises a prostate cancer cell. In some embodiments, the sample is selected from the group consisting of blood, serum, plasma, urine, prostatic fluid and semen. In some embodiments, the detecting comprises contacting said sample with reagents that specifically bind to said one or more lipids. In some embodiments, the reagents are antibodies. In some embodiments, the one or more lipids comprises three or more lipids. In some embodiments, the one or more lipids comprise HexCer, LacCer, Gb3, GM1, GM2, GM3, and GD1 and combinations thereof.

In some embodiments, the present invention provides kits comprising: a) one or more reagents for detecting the presence of one or more lipids in an extracellular vesicle; and b) a reference for correlating the presence or amount of said two or more lipids with the presence of prostate cancer cells. In some embodiments, the reference comprises a reference sample. In some embodiments, the reference is selected from the group consisting of a computer readable medium comprising an algorithm and graphic materials. In some embodiments, the reagents comprise antibodies. In some embodiments, the reagents comprise one or more antibodies specific for one or more of glycosphingolipids, sphingomyelin and cholesterol. In some embodiments, the glycosphingolipids are selected from the group consisting of HexCer, LacCer, Gb3, GM1, GM2, GM3, and GD1.

In some embodiments, the present invention provides for the use of reagents for detecting the presence or amount of one or more lipids in a biological sample for providing a diagnosis or prognosis related to prostate cancer. In some embodiments, the reagents are antibodies. In some embodiments, the antibodies are specific for two or more of glycosphingolipids, sphingomyelin and cholesterol. In some embodiments, the glycosphingolipids are selected from the group consisting of HexCer, LacCer, Gb3, GM1, GM2, GM3, and GD1.

Additional embodiments are described herein.

### DESCRIPTION OF THE FIGURES

FIG. 1. This figure shows the number of times that lipids are increased in vesicles in relation to cells in nmol/mg protein.

FIG. 2A. This figure shows the number of times that specific lipid classes are increased in vesicles in relation to cells in mol %. B. This figure shows the number of times that specific lipids are increased in cells in relation to vesicles in mol %.

### DEFINITIONS

To facilitate an understanding of the present invention, a number of terms and phrases are defined below:

As used herein, the terms “detect”, “detecting” or “detection” may describe either the general act of discovering or discerning or the specific observation of a detectably labeled composition.

As used herein, the term “subject” refers to any organisms that are screened using the diagnostic methods described herein. Such organisms preferably include, but are not limited to, mammals (e.g., murines, simians, equines, bovines, porcines, canines, felines, and the like), and most preferably includes humans.

The term “diagnosed,” as used herein, refers to the recognition of a disease by its signs and symptoms, or genetic analysis, pathological analysis, histological analysis, and the like.

A “subject suspected of having cancer” encompasses an individual who has received an initial diagnosis (e.g., a CT scan showing a mass or increased PSA level) but for whom the stage of cancer or presence or absence of lipid markers indicative of cancer is not known. The term further includes people who once had cancer (e.g., an individual in remission). In some embodiments, “subjects” are control subjects that are suspected of having cancer or diagnosed with cancer.

As used herein, the term “characterizing cancer in a subject” refers to the identification of one or more properties of a cancer sample in a subject, including but not limited to, the presence of benign, pre-cancerous or cancerous tissue, the stage of the cancer, and the subject's prognosis. Cancers may be characterized by the identification of the expression of one or more lipids in cancer cells.

As used herein, the term “characterizing a prostate sample in a subject” refers to the identification of one or more properties of a prostate tissue sample (e.g., including but not limited to, the presence of cancerous tissue, the presence or absence of specific lipids, the presence of pre-cancerous tissue that is likely to become cancerous, and the presence of cancerous tissue that is likely to metastasize). In some embodiments, tissues are characterized by the identification of one or more lipids, including but not limited to, the cancer markers disclosed herein.

As used herein, the term “stage of cancer” refers to a qualitative or quantitative assessment of the level of advancement of a cancer. Criteria used to determine the stage of a cancer include, but are not limited to, the size of the tumor and the extent of metastases (e.g., localized or distant).

As used herein, the term “purified” or “to purify” refers to the removal of components (e.g., contaminants) from a sample.

As used herein, the term “sample” is used in its broadest sense. In one sense, it is meant to include a specimen or culture obtained from any source, as well as biological and environmental samples. Biological samples may be obtained from animals (including humans) and encompass fluids, solids, and tissues. Biological samples include blood products, such as plasma, serum and the like. Such examples are not however to be construed as limiting the sample types applicable to the present invention.

### DETAILED DESCRIPTION OF THE INVENTION

The present invention relates to compositions and methods for cancer diagnosis, research and therapy, including but not limited to, cancer markers. In particular, the present invention relates to lipids as diagnostic markers for prostate cancer.

Cells are able to release vessicles (1-4). Extracellular vesicles contain a variety of proteins, lipids and nucleic acids. Proteomic studies have shown that extracellular vesicles contain several hundred proteins (5). A set of these proteins (CD63, Tsg101, alix, CD9) is commonly found in vesicles released from different cell types, but extracellular vesicles also contain proteins that are specific to the parent cells from which they are released. This, together with the fact that extracellular vesicles can reach biological fluids, has led to the idea of using extracellular vesicles as a source for biomarkers (6-10). Concerning cancer, promising results show that plasma samples from ovarian cancer patients contain claudin-4-containing exosomes (11) and that plasma from melanoma patients contain high levels of exosomes expressing CD63 and caveolin-1 (12).

In contrast to the extensive information about protein composition of extracellular vesicles, much less is known about their lipid composition and only a few studies have been published (13-15). It has been shown that although the lipid composition in various exosome populations differs, extracellular vesicles are often enriched in cholesterol and SM and have an increased amount of saturated fatty acids, suggesting that the membranes of extracellular vesicles contain lipid raft-like domains (16).

Knowledge about the lipid composition of exosomes may provide new biomarkers for disease. In order to obtain such information, a quantitative molecular lipidomic analysis was performed. In particular, lipid profiles of the prostate cancer cell line PC-3 and of the vesicles released by this cell line were determined. The lipidomic approach used in this study provided an extensive lipidomic read-out of the samples analyzed including the concentrations of 24 lipid classes. Major differences in the lipid classes were found between the extracellular vesicles and the parent cells. The results presented here constitute the most detailed description of the lipid composition of an extracellular vesicles membrane to date. Experiments conducted during the course of development of embodiments of the present invention demonstated that extracellular vesicles released by PC-3 cells were highly enriched in glycosphingolipids, sphingomyelin and cholesterol, but contained much less phosphatidylinositol, phosphatidylglycerol and phosphatidylcholine (mol % of total lipids). Furthermore, the molecular lipid composition of phosphatidylserine was very different in extracellular vesicles versus cells.

Accordingly, embodiments of the present invention provide compositions and methods for the diagnosis, screening, and research of prostate cancer comprising the detection and analysis of lipids in extracellular vesicles. In some embodiments, the presence of extracellular vesicles comprising a variety of lipids is detected. In other embodiments, the specific lipids in an extracellular vesicle are detected (e.g., extracellular vesicles isolated from prostate cell samples or biopsies).

The present invention is not limited to the detection of a particular lipid. Examples include, but are not limited to, those described in Table 1. Specific examples include, but are not limited to, glycosphingolipids (e.g., HexCer, LacCer, Gb3, GM1, GM2, GM3, and GD1), sphingomyelin, and cholesterol.

In some embodiments, lipids that are differentially found in extracellular vesicles of prostate cancer cells, but not normal prostate cells are detected. For example, in some embodiments, the presence of such lipids in a sample is indicative of a diagnosis of prostate cancer in a subject.

In some embodiments, lipids are measured in an extracellular vesicle sample from a subject. Examples of suitable samples include, but are not limited to, prostatic secretions or a fraction thereof (e.g., plasma, serum, urine, urine supernatant, urine cell pellet or prostate cells). A urine sample is preferably collected immediately following an attentive digital rectal examination, which causes prostate cells from the prostate gland to shed into the urinary tract.

In some embodiments, samples are processed to enrich for prostate cells or extracellular vesicles prior to analysis. A variety of techniques known to those of ordinary skill in the art may be used for this purpose, including but not limited: centrifugation; immunocapture; cell lysis; and, magnetic capture. Commercial kits for isolation of such microvesicles are available (e.g., Exoquick from SBI Systems Biosciences and Exotest from Hansabiomed).

In some embodiments, analytical techniques are utilized to determine the lipid content or lipid profile of an extracellular vesicle sample from patient. In some embodiments, the lipid content or profile of the patient extracellular vesicle sample is compared to the lipid content or profile of a reference sample or standard. In some embodiments, an altered lipid content or profile in the patient sample as compared to the reference is indicative of or correlates to the presence of a disease or condition in the patient, for example, the presence of a tumor or prostate cancer. In some embodiments, elevated amounts of one or more of glycosphingolipids, sphingomyelin and cholesterol in the patient sample as compared to the reference is indicative of prostate cancer. In some embodiments, lowered amounts of one or more of phosphatidylinositol, phosphatidylglycerol and phosphatidylcholine as compared to the reference is indicative of prostate cancer. In some embodiments, elevated amounts of one or more of glycosphingolipids, sphingomyelin and cholesterol in the patient sample as compared to the reference and lowered amounts of one or more of phosphatidylinositol, phosphatidylglycerol and phosphatidylcholine as compared to the reference is indicative of prostate cancer. In some embodiments, a lipid profile comprising one, two, three, four five or more lipids as described above is determined for a sample from a subject and compared to a reference lipid profile.

The present invention is not limited to a particular lipid detection method. Any suitable method may be utilized. In some embodiments, antibodies specific for the particular lipid to be detected are utilized. In some embodiments, ELISA or another immunoassay (e.g., an automated assay) is utilized to detect antibody binding to lipids.

Antibodies useful determining the lipid profile of extracellular vesicles may be any monoclonal or polyclonal antibody, as long as it can recognize, preferably specifically, the lipid. Antibodies can be produced by using lipids as the antigen according to known antibody or antiserum preparation processes. Anti-lipid antibodies are also commercially available from several sources. See, for example, Horkko et al., J. Clin. Invest. 98:815-825, 1996; Palinski et al., J. Clin. Invest. 98:800-814, 1996; O'Brien et al., J. Lipid Res. 50:2245-57, 2009; Schuster, J. immunol. 122(3):900-905, 1979; Rapport and Graf, Cancer 8(3):538-45, 1955; Young et al., J. Biol. Chem., 256(21):10967-72, 1981; Kannagi, Methods Enzymol., 312:160-79, 2000.

An ELISA, short for Enzyme-Linked ImmunoSorbent Assay, is a biochemical technique to detect the presence of an antibody or an antigen in a sample. It utilizes a minimum of two antibodies, one of which is specific to the antigen and the other of which is coupled to an enzyme. The second antibody will cause a chromogenic or fluorogenic substrate to produce a signal. Variations of ELISA include sandwich ELISA, competitive ELISA, and ELISPOT. Because the ELISA can be performed to evaluate either the presence of antigen or the presence of antibody in a sample, it is a useful tool both for determining serum antibody concentrations and also for detecting the presence of antigen.

In some embodiments, flow cytometry is utilized to separate extracellular vesicles or identify lipid on extracellular vesicles. Flow cytometry is a technique for counting, examining and sorting microscopic particles suspended in a stream of fluid. It allows simultaneous multiparametric analysis of the physical and/or chemical characteristics of single cells flowing through an optical/electronic detection apparatus. A beam of light (e.g., a laser) of a single frequency or color is directed onto a hydrodynamically focused stream of fluid. A number of detectors are aimed at the point where the stream passes through the light beam; one in line with the light beam (Forward Scatter or FSC) and several perpendicular to it (Side Scatter (SSC) and one or more fluorescent detectors). Each suspended particle passing through the beam scatters the light in some way, and fluorescent chemicals in the particle may be excited into emitting light at a lower frequency than the light source. The combination of scattered and fluorescent light is picked up by the detectors, and by analyzing fluctuations in brightness at each detector, one for each fluorescent emission peak, it is possible to deduce various facts about the physical and chemical structure of each individual particle. FSC correlates with the cell volume and SSC correlates with the density or inner complexity of the particle (e.g., shape of the nucleus, the amount and type of cytoplasmic granules or the membrane roughness).

In some embodiments, mass spectrometry (See e.g., example 1) is utilized for determining the lipids in extracellular vesicles. Suitable mass spectrometry techniques include, but are not limited to LC-MS, LC-MS/MS, UHPLC-AS and UHPLC-MS/MS. The techniques may optionally be utilized with multiple reaction monitoring (MRM) in positive or negative ion modes. In some embodiments, gas chromatography or liquid chromatography are utilized for determining the lipids in extracellular vesicles.

In some embodiments, molecules (e.g., proteins that are not antibodies) bind to lipids and are then detected using a suitable analytical method (e.g., immunoassay or other analytical method).

In some embodiments, a computer-based analysis program is used to translate the raw data generated by the detection assay (e.g., the presence, absence, or amount of a given marker or markers, preferably lipid markers) into data of predictive value for a clinician. The clinician can access the predictive data using any suitable means. Thus, in some preferred embodiments, the present invention provides the further benefit that the clinician, who is not likely to be trained in genetics or molecular biology, need not understand the raw data. The data is presented directly to the clinician in its most useful form. The clinician is then able to immediately utilize the information in order to optimize the care of the subject.

The present invention contemplates any method capable of receiving, processing, and transmitting the information to and from laboratories conducting the assays, information provides, medical personal, and subjects. For example, in some embodiments of the present invention, a sample (e.g., a biopsy or a serum or urine sample) is obtained from a subject and submitted to a profiling service (e.g., clinical lab at a medical facility, genomic profiling business, etc.), located in any part of the world (e.g., in a country different than the country where the subject resides or where the information is ultimately used) to generate raw data. Where the sample comprises a tissue or other biological sample, the subject may visit a medical center to have the sample obtained and sent to the profiling center, or subjects may collect the sample themselves (e.g., a urine sample) and directly send it to a profiling center. Where the sample comprises previously determined biological information, the information may be directly sent to the profiling service by the subject (e.g., an information card containing the information may be scanned by a computer and the data transmitted to a computer of the profiling center using an electronic communication system). Once received by the profiling service, the sample is processed and a profile is produced (i.e., lipid profile data) specific for the diagnostic or prognostic information desired for the subject.

The profile data is then prepared in a format suitable for interpretation by a treating clinician. For example, rather than providing raw expression data, the prepared format may represent a diagnosis or risk assessment (e.g., presence or absence of a lipid) for the subject, along with recommendations for particular treatment options. The data may be displayed to the clinician by any suitable method. For example, in some embodiments, the profiling service generates a report that can be printed for the clinician (e.g., at the point of care) or displayed to the clinician on a computer monitor.

In some embodiments, the information is first analyzed at the point of care or at a regional facility. The raw data is then sent to a central processing facility for further analysis and/or to convert the raw data to information useful for a clinician or patient. The central processing facility provides the advantage of privacy (all data is stored in a central facility with uniform security protocols), speed, and uniformity of data analysis. The central processing facility can then control the fate of the data following treatment of the subject. For example, using an electronic communication system, the central facility can provide data to the clinician, the subject, or researchers.

In some embodiments, the subject is able to directly access the data using the electronic communication system. The subject may chose further intervention or counseling based on the results. In some embodiments, the data is used for research use. For example, the data may be used to further optimize the inclusion or elimination of markers as useful indicators of a particular condition or stage of disease or as a companion diagnostic to determine a treatment course of action.

### EXPERIMENTAL

The following examples are provided in order to demonstrate and further illustrate certain preferred embodiments and aspects of the present invention and are not to be construed as limiting the scope thereof.

### Example 1

### A. Methods

### Abbreviations

Ceramides (Cer), cholesteryl esters (CE), diacylglycerols (DAG), free cholesterol (FC), ganglioside GD1 (GD1), ganglioside GM1 (GM1), ganglioside GM2 (GM2), ganglioside GM3 (GM3), globotriaosylceramides, (Gb₃), glycosphingolipids (GSL), hexosylceramides (HexCer), lactosylceramides (LacCer), lysophosphatidylethanolamines (LPE), lysophosphatidylinositols (LPI), mass spectrometry (MS), phosphatidic acids (PA), phosphatidylcholines (PC), phosphatidylcholines with ether-linked alkenyl (PCP), phosphatidylcholines with ether linked alkyl (PCO), phosphatidylethanolamines (PE), phosphatidylethanolamines with ether-linked alkenyl (PEP), phosphatidylethanolamines with ether-linked alkyl (PEO), phosphatidylglycerols (PG), phosphatidylinositols (PI), phosphatidylserines (PS), sphingomyelins (SM).

### Materials

DMEM/F-12 (1:1 Mix of DMEM and Ham's F-12) medium and keratinocyte-serum free medium kit with L-glutamine, epidermal growth factor and bovine pituitary extract were from Gibco, Invitrogen Dynal, Norway. Bicinchoninic acid proteins assay kit was from Pierce, Thermo Scientific, Rockford, Ill. Mini EDTA-free Protease Inhibitor Cocktail Tablets were purchased from Roche Diagnostics, Oslo, Norway.

### Cell Culture

The epithelial human prostate cancer cell line PC-3 (17) obtained from the American Type Culture Collection was maintained in a 1:1 mixture of Ham's F12 medium and Dulbecco's modified Eagle's medium supplemented with 7% foetal calf serum, 100 units/ml penicillin and 100 μg/ml streptomycin. The epithelial human prostate cell line RWPE-1 was obtained from the American Type Culture Collection and grown in keratinocyte serum-free medium supplemented with bovine pituitary extract (0.05 mg/ml) and EGF (5 ng/ml), 100 units/ml penicillin, and 100 μg/ml streptomycin. Cells were maintained at 37° C. in an atmosphere of 5% CO₂/95% air.

### Microvesicle Isolation

PC-3 cells and RWPE-1 cells were grown in serum-free cell culture medium for 3 days and exosomes were isolated from the culture medium. The culture medium was centrifuged to remove cell debris at 300 g for 10 min, 1,000 g for 10 min and at 10,000 g for 30 min. Vesicles were then collected by ultracentrifugation at 100,000 g for 70 min first in a Ti70 rotor and then in a SW40 rotor. The vesicles were then washed with a large volume of phosphate-buffered saline (PBS), and then pelleted again by ultracentrifugation at 100,000 g for 70 min in a SW40 rotor. The vesicles were solubilized in PBS and stored at −80° C. prior to analysis.

### Cell Collection

Cells were trypsinized and centrifuged at 300 g for 10 min. Cells were then washed with PBS at 4° C. and centrifuged again. The cell pellets were stored at −80° C. prior to analysis.

### Cell Lysates

Cells were lysed at 4° C. in a lysis buffer containing 50 mM Tris-HCl, 0.3 M NaCl, 1 mM EDTA and 0.5% Triton X-100, pH 7.4, in the presence of a protease inhibitor mixture. Insoluble material was removed by centrifugation at 20,000 g for 10 min at 4° C.

### Protein Determination

Cells and vesicles were lysed in the presence of a protease inhibitor mixture. Then, the protein content was determined using a bicinchoninic acid protein assay kit according to the manufacturer's instructions. Bovine serum albumin was use as standard protein.

### Lipid Extraction

Lipid extraction was carried out in a Microlab Star Robot (Hamilton Robotics). Lipids were extracted using a modified Folch lipid extraction (18). After lipid extraction, samples were reconstituted in chloroform:methanol (1:2, v/v) For quantification of free cholesterol, an aliquot of each lipid extract was treated with acetyl chloride to derivatize FC to modified cholesteryl ester species according to Liebisch et al. (19).

The lipid extracts for shotgun analysis were analyzed on a hybrid triple quadrupole/linear ion trap mass spectrometer (QTRAP 5500, AB SCIEX) equipped with a robotic nanoflow ion source (NanoMate HD, Advion Biosciences) (20). Sphingolipids were analyzed on a hybrid triple quadrupole/linear ion trap mass spectrometer (4000 Q TRAP, AB SCIEX) equipped with an ultra high pressure liquid chromatography (UHPLC) system (CTC HTC PAL autosampler, CTC Analytics AG and Rheos Allegro pump, Flux instruments) using in-house developed methods based on multiple reaction monitoring (MRM) in positive or negative ion modes.

### Data Processing

The following software was used in the data analysis: LipidProfiler (version 1.0.99) for processing of QTRAP 5500 data, MultiQuant (version 2.0) for processing of 4000 Q TRAP data, Excel (version 2007), Oracle Database (version 11.1.0.6.0), SAS (version 9.2), Tableau Desktop (version 6.0).

### B. Results

### Isolation of Extracellular Vesicles

Released vesicles were isolated as previously described (21) but, in order to accumulate a large amount of vesicles, cells were incubated for 3 days before vesicles were collected from the culture medium. Typically 0.78±0.13 (SD, n=4) μg of exosome protein per million of PC-3 cells were obtained. For lipidomic analysis vesicles released from approximately 400 million cells isolated in four independent experiments were mixed. Exosomes were also isolated from the non cancerous prostate epithelial cell line RWPE-1. The amount of protein associated with exosomes released from these cells was 0.06±0.03 (SD, n=4) μg of exosome protein per million RWPE-1 cells, e.g., approximately 8% of the amount released from PC-3 cells, and too low to perform a complete lipidomic analysis.

### Analysis of Lipid Classes

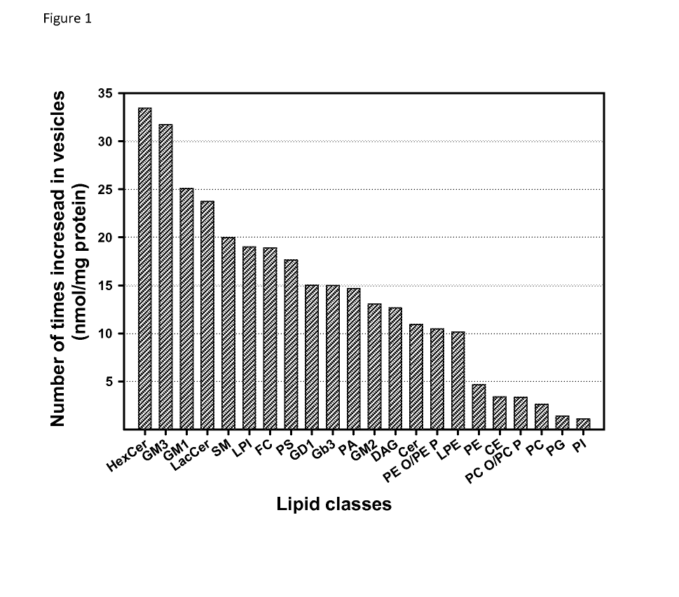

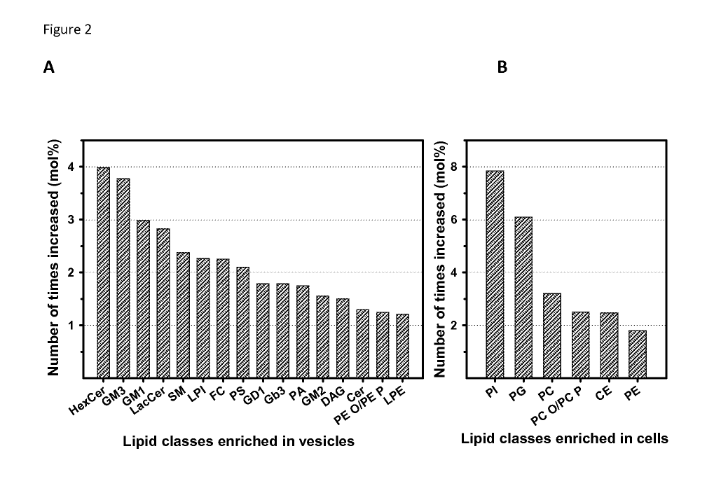

In order to quantify as many lipid species as possible both shotgun lipidomics and sphingolipid lipidomics were performed. The results obtained following analysis of three samples are presented; two independent experiments performed with PC-3 cells and extracellular vesicles showed similar results. The analyses revealed molecular lipids species of 24 lipid classes. The data are presented both as nmol/mg of protein and as mol % of total lipids in Table 1. The total concentration of lipids was 101.7±9.3 (SD, n=3) nmol/mg protein in PC-3 cells and 812.5±73.1 (SD, n=3) nmol/mg protein in extracellular vesicles, thus showing that exosomes contain 8 times higher amounts of lipids per protein than PC-3 cells. Most lipid classes (measured as nmol/mg protein) were elevated inextracellular vesicles, and the concentration of some lipid classes such as HexCer, LacCer, SM, FC, GM1, GM3, GD1 and Gb3 was more than 15 times higher in extracellular vesicles (FIG. 1). Since the total concentration of lipids is very different in vesicles and in parent cells, the data were also shown as percentage of the total amount of lipids present in the samples (mol %) (FIG. 2). This analysis revealed that extracellular vesicles (FIG. 2A) are highly enriched in GSL, FC and SM, whereas the mol % of e.g. PC, PI, PG, PE and CE is higher in cells than in extracellular vesicles (Table 1 and FIG. 2B). The percentage of several lipid classes such as PE, PS, PC 0, PE P, LPE and Cer was, however, quite similar in cells and extracellular vesicles (Table 1 and FIG. 2). These data show that there is a large increase in GSL content in the extracellular vesicles compared to the parent cells. The lipid profiles PC-3 and RWPE-1 cells were quite similar (data not shown).

TABLE 1
Summary data for the lipid classes quantified in PC-3 cells and exosomes.

| | Exosomes | Cells | Exosomes | Cells |
|---|---|---|---|---|
| LIPID CLASS | (nmol/mg protein) | (nmol/mg protein) | (mol %) | (% mol) |
| FC | 342.39 ± 7.09 | 18.12 ± 1.51 | 43.52 ± 3.97 | 19.25 ± 0.97 |
| SM | 128.88 ± 17.26 | 6.45 ± 0.32 | 16.26 ± 1.11 | 6.87 ± 0.55 |
| PC | 121.45 ± 20.86 | 46.27 ± 5.96 | 15.28 ± 1.39 | 49.06 ± 3.27 |
| PS | 92.49 ± 13.49 | 5.24 ± 1.14 | 11.66 ± 0.69 | 5.54 ± 0.94 |
| PE | 46.07 ± 11.67 | 9.88 ± 1.33 | 5.78 ± 0.96 | 10.59 ± 20 |
| PE O + PE P | 26.06 ± 5.56 | 2.49 ± 0.30 | 3.27 ± 0.42 | 2.67 ± 0.46 |
| DAG | 11.88 ± 1.13 | 0.94 ± 0.02 | 1.52 ± 0.26 | 1.00 ± 0.08 |
| PC O + PC P | 6.46 ± 0.95 | 1.92 ± 0.30 | 0.81 ± 0.05 | 2.04 ± 0.19 |
| HexCer | 6.02 ± 0.42 | 0.18 ± 0.03 | 0.76 ± 0.04 | 0.20 ± 0.03 |
| Cer | 2.51 ± 0.06 | 0.23 ± 0.03 | 0.32 ± 0.02 | 0.24 ± 0.02 |
| PG | 1.34 ± 0.63 | 0.97 ± 0.09 | 0.17 ± 0.07 | 1.03 ± 0.10 |
| PA | 1.32 ± 0.09 | 0.09 ± 0.02 | 0.16 ± 0.00 | 0.09 ± 0.02 |
| PI | 1.04 ± 0.20 | 0.97 ± 0.03 | 0.13 ± 0.01 | 1.03 ± 0.10 |
| LacCer | 0.95 ± 0.03 | 0.04 ± 0.00 | 0.12 ± 0.01 | 0.04 ± 0.00 |
| LPI | 0.76 ± 0.43 | 0.04 ± 0.02 | 0.09 ± 0.05 | 0.04 ± 0.01 |
| LPE | 0.71 ± 0.09 | 0.07 ± 0.01 | 0.09 ± 0.00 | 0.07 ± 0.01 |
| CE | 0.68 ± 0.47 | 0.20 ± 0.04 | 0.08 ± 0.06 | 0.21 ± 0.04 |
| Gb3 | 0.15 ± 0.01 | 0.01 ± 0.00 | 0.02 ± 0.00 | 0.01 ± 0.00 |

### REFERENCES

1. Ludwig, A. K., and Giebel, B. (2012) Exosomes: Small vesicles participating in intercellular communication. Int. J. Biochem. Cell Biol. 44, 11-15
2. Tual-Chalot, S., Leonetti, D., Andriantsitohaina, R., and Martinez, M. C. (2011) Microvesicles: intercellular vectors of biological messages. Mol. Interv. 11, 88-94
3. Lee, T. H., D'Asti, E., Magnus, N., Al-Nedawi, K., Meehan, B., and Rak, J. (2011) Microvesicles as mediators of intercellular communication in cancer—the emerging science of cellular ‘debris’. Semin. Immunopathol. 33, 455-467
4. Eldh, M., Ekstrom, K., Valadi, H., Sjostrand, M., Olsson, B., Jernas, M., and Lotvall, J. (2010) Exosomes communicate protective messages during oxidative stress; possible role of exosomal shuttle RNA. PLoS. One. 5, e15353
5. Mathivanan, S., Fahner, C. J., Reid, G. E., and Simpson, R. J. (2011) ExoCarta 2012: database of exosomal proteins, RNA and lipids. Nucleic Acids Res.
6. Duijvesz, D., Luider, T., Bangma, C. H., and Jenster, G. (2011) Exosomes as biomarker treasure chests for prostate cancer. Eur. Urol. 59, 823-831
7. Michael, A., Bajracharya, S. D., Yuen, P. S., Zhou, H., Star, R. A., Illei, G. G., and Alevizos, I. (2010) Exosomes from human saliva as a source of microRNA biomarkers. Oral Dis. 16, 34-38
8. Roberson, C. D., Atay, S., Gercel-Taylor, C., and Taylor, D. D. (2010) Tumor-derived exosomes as mediators of disease and potential diagnostic biomarkers. Cancer Biomark. 8, 281-291
9. Miranda, K. C., Bond, D. T., McKee, M., Skog, J., Paunescu, T. G., Da, S. N., Brown, D., and Russo, L. M. (2010) Nucleic acids within urinary exosomes/microvesicles are potential biomarkers for renal disease. Kidney Int.
10. Raimondo, F., Morosi, L., Chinello, C., Magni, F., and Pitto, M. (2011) Advances in membranous vesicle and exosome proteomics improving biological understanding and biomarker discovery. Proteomics. 11, 709-720
11. Li, J., Sherman-Baust, C. A., Tsai-Turton, M., Bristow, R. E., Roden, R. B., and Morin, P. J. (2009) Claudin-containing exosomes in the peripheral circulation of women with ovarian cancer. BMC. Cancer. 9:244,
12. Logozzi, M., De, M. A., Lugini, L., Borghi, M., Calabro, L., Spada, M., Perdicchio, M., Marino, M. L., Federici, C., Iessi, E. and others (2009) High levels of exosomes expressing CD63 and caveolin-1 in plasma of melanoma patients. PLoS. One. 4, e5219
13. Subra, C., Laulagnier, K., Perret, B., and Record, M. (2007) Exosome lipidomics unravels lipid sorting at the level of multivesicular bodies. Biochimie. 89, 205-212
14. Arienti, G., Carlini, E., Polci, A., Cosmi, E. V., and Palmerini, C. A. (1998) Fatty acid pattern of human prostasome lipid. Arch. Biochem. Biophys. 358, 391-395
15. Arvidson, G., Ronquist, G., Wikander, G., and Ojteg, A. C. (1989) Human prostasome membranes exhibit very high cholesterol/phospholipid ratios yielding high molecular ordering. Biochim. Biophys. Acta. 984, 167-173
16. de, G. A., Geminard, C., Fevrier, B., Raposo, G., and Vidal, M. (2003) Lipid raft-associated protein sorting in exosomes. Blood. 102, 4336-4344
17. Kaighn, M. E., Narayan, K. S., Ohnuki, Y., Lechner, J. F., and Jones, L. W. (1979) Establishment and characterization of a human prostatic carcinoma cell line (PC-3). Invest Urol. 17, 16-23
19. Liebisch, G., Binder, M., Schifferer, R., Langmann, T., Schulz, B., and Schmitz, G. (2006) High throughput quantification of cholesterol and cholesteryl ester by electrospray ionization tandem mass spectrometry (ESI-MS/MS). Biochim. Biophys. Acta. 1761, 121-128
20. Stahlman, M., Ejsing, C. S., Tarasov, K., Perman, J., Boren, J., and Ekroos, K. (2009) High-throughput shotgun lipidomics by quadrupole time-of-flight mass spectrometry. J. Chromatogr. B Analyt. Technol. Biomed. Life Sci. 877, 2664-2672
21. Llorente, A., van, D. B., and Sandvig, K. (2007) Cholesterol regulates prostasome release from secretory lysosomes in PC-3 human prostate cancer cells. Eur. J. Cell Biol. 86, 405-415

All publications, patents, patent applications and accession numbers mentioned in the above specification are herein incorporated by reference in their entirety. Although the invention has been described in connection with specific embodiments, it should be understood that the invention as claimed should not be unduly limited to such specific embodiments. Indeed, various modifications and variations of the described compositions and methods of the invention will be apparent to those of ordinary skill in the art and are intended to be within the scope of the following claims.

1. A method for predicting a predisposition to prostate cancer in a subject, diagnosing a prostate cancer in a subject, predicting the likelihood of recurrence of prostate cancer in a subject, providing a prognosis for a subject with prostate cancer, or selecting a subject with cervical cancer for treatment with a particular therapy, comprising:
(a) detecting the presence of one or more lipids in a sample from said subject using an in vitro assay and
(b) comparing said one or more lipids in said sample with a reference, wherein an altered amount of said one or more lipids relative to said reference provides an indication selected from the group consisting of an indication of a predisposition of the subject to prostate cancer, an indication that the subject has prostate cancer, an indication of the likelihood of recurrence of the prostate cancer in the subject, an indication of survival of the subject, an indication of the likely outcome of treatment of the prostate cancer and an indication that the subject is a candidate for treatment with a particular therapy.

2. The method of claim 1, wherein elevated amounts of one or more of glycosphingolipids, sphingomyelin, or cholesterol, in the biological sample as compared to a reference is indicative of prostate cancer.

3. The method of claim 1, wherein said glycosphingolipids are selected from the group consisting of HexCer, LacCer, Gb3, GM1, GM2, GM3, and GD1.

4. The method of claim 1, wherein lowered amounts of one or more of phosphatidylinositol, phosphatidylglycerol and phosphatidylcholine in the biological sample as compared to a reference is indicative of prostate cancer.

5. The method of claim 1, wherein said in vitro assay is selected from the group consisting of a mass spectrometry assay, a gas chromatography assay, a liquid chromatography assay, and immunoassay, and combinations thereof.

6. The method of claim 1, wherein said method further comprises the step of enriching said sample for the presence of extracellular vesicles.

7. The method of claim 1, wherein said sample comprises a prostate cancer cell.

8. The method of claim 1, wherein said sample is selected from the group consisting of blood, serum, plasma, urine, prostatic fluid and semen.

9. The method of claim 1, wherein said detecting comprises contacting said sample with reagents that specifically bind to said one or more lipids.

10. The method of claim 1, wherein said reagents are antibodies.

11. The method of claim 1, wherein said one or more lipids comprises three or more lipids.

12. The method of claim 1, wherein said one or more lipids comprises HexCer, LacCer, Gb3, GM1, GM2, GM3, and GD1.

13. The method of claim 1, wherein said assay is a mass spectrometry assay.

14. A method of screening for the presence of prostate cancer in a subject, comprising
(a) providing a biological sample from a subject and
(b) analyzing said sample for the presence or amount of one or more lipids in said sample, wherein the presence of said lipids in said sample is indicative of prostate cancer in said subject.

15. The method of claim 14, wherein elevated amounts of one or more of glycosphingolipids, sphingomyelin and cholesterol in the biological sample as compared to a reference is indicative of prostate cancer.

16. The method of claim 14, wherein said glycosphingolipids are selected from the group consisting of HexCer, LacCer, Gb3, GM1, GM2, GM3, and GD1.

17. The method of claim 14, wherein lowered amounts of one or more of phosphatidylinositol, phosphatidylglycerol and phosphatidylcholine in the biological sample as compared to a reference is indicative of prostate cancer.

18. The method of claim 14, wherein said in vitro assay is selected from the group consisting of a mass spectrometry assay, a gas chromatography assay, a liquid chromatography assay, and immunoassay, and combinations thereof.

19. The method of claim 14, wherein said method further comprises the step of enriching said sample for the presence of extracellular vesicles.

20. The method of claim 14, wherein said sample is selected from the group consisting of blood, serum, plasma, urine, prostatic fluid and semen.

21. The method of claim 14, wherein said detecting comprises contacting said sample with reagents that specifically bind to said one or more lipids.

22. The method of claim 14, wherein said reagents are antibodies.

23. The method of claim 14, wherein said one or more lipids comprises two or more lipids.

24. The method of claim 14, wherein said one or more lipids comprises three or more lipids.

25. The method of claim 14, wherein said one or more lipids comprises HexCer, LacCer, Gb3, GM1, GM2, GM3, and GD1.

26. A kit comprising:
a) one or more reagents for detecting the presence of one or more lipids in an extracellular vesicle; and
b) a reference for correlating the presence or amount of said two or more lipids with the presence of prostate cancer cells.

27. The kit of claim 26, wherein said reference comprises a reference sample.

28. The kit of claim 26, wherein said reference is selected from the group consisting of a computer readable medium comprising an algorithm and graphic materials.

29. The kit of claim 26, wherein said reagents comprise antibodies.

30. The kit of claim 29, wherein said reagents comprise one or more antibodies specific for one or more of glycosphingolipids, sphingomyelin and cholesterol.

31. The kit of claim 30, wherein said glycosphingolipids are selected from the group consisting of HexCer, LacCer, Gb3, GM1, GM2, GM3, and GD1.

32-35. (canceled)
